(19) United States
(12) Patent Application Publication
(10) Pub. No.: US 2021/0265050 A1
(43) Pub. Date: Aug. 26, 2021

(54) DISCHARGE CARE PLAN TAILORING FOR IMPROVING KPIS

(71) Applicant: KONINKLIJKE PHILIPS N.V., EINDHOVEN (NL)

(72) Inventors: Eran SIMHON, Boston, MA (US); Reza SHARIFI SEDEH, Malden, MA (US)

(21) Appl. No.: 17/253,387

(22) PCT Filed: Jun. 19, 2019
(86) PCT No.: PCT/EP2019/066211

§ 371 Date: Dec. 17, 2020

Related U.S. Application Data
(60) Provisional application No. 62/690,692, filed on Jun. 27, 2018.

(51) Int. Cl. G16H 40/20; G06Q 10/06; G06Q 30/02

(52) U.S. Cl. CPC G16H 40/20; G06Q 30/0283; G06Q 10/06393

(57) ABSTRACT

A care plan system, including: a key performance indicator (KPI) model configured to predict the value of a KPI for a specific patient based upon patient data and care plan elements; cost data indicating the cost of the care plan elements; a graphical user interface (GUI) configured to receive first suggested care plan elements, to provide a first predicted value of the KPI and a first care plan cost associated with the first suggested care plan elements using the KPI model, and to present the first predicted KPI value and first care plan cost.

### TECHNICAL FIELD

Various exemplary embodiments disclosed herein relate generally to discharge care plan tailoring for improving key performance indicators (KPIs).

### BACKGROUND

Transitioning from a fee for service model to value-based care has forced healthcare systems to focus more on the entire life of their patient beyond hospital walls. In order to manage patient health journey outside the hospital, healthcare professionals are providing care plans to their patients. Care plans provide direction for individualized care of patients. Ideally, a care plan should flow from each patient's unique list of diagnoses and should be organized by the individual's specific needs to guarantee continuity of care.

The care plan is a means of communicating and organizing the actions of a constantly changing medical staff. In order to provide the best experience of care, health care professionals must tailor services to recognize patients as individuals and to respond to their needs, preferences, and values, taking into account both shared requirements and individual characteristics such as, for example, individuals' expectations of service and preferences, their cultural background, age, and gender.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a care plan system, including: a key performance indicator (KPI) model configured to predict the value of a KPI for a specific patient based upon patient data and care plan elements; cost data indicating the cost of the care plan elements; and a graphical user interface (GUI) configured to receive first suggested care plan elements, to provide a first predicted value of the KPI and a first care plan cost associated with the first suggested care plan elements using the KPI model, and to present the first predicted KPI value and first care plan cost.

Various embodiments are described, wherein the KPI model is a linear or logistic regression model.

Various embodiments are described, wherein the GUI presents a plot of cost versus the predicted KPI value for the first predicted KPI and first cost.

Various embodiments are described, wherein the GUI is configured to receive second suggested care plan elements, to provide a second predicted value of the KPI and a second care plan cost associated with the second suggested care plan elements using the KPI model, to present the second predicted KPI value and second care plan cost, and to add the second predicted KPI value and the second cost to the plot.

Various embodiments are described, further including a method for generating care plan, including: receiving, via a graphical user interface (GUI), first care plan elements; predicting, by a processor, a first value of a key performance indicator (KPI) using a KPI model for a specific patient based upon patient data and the first care plan elements; producing first cost data indicating the cost of the care plan elements; and presenting the first predicted KPI value and first care plan cost the on the GUI.

Various embodiments are described, wherein the KPI model is a linear or logistic regression model.

Various embodiments are described, further including presenting by the GUI a plot of cost versus the predicted KPI value for the first predicted KPI and first cost.

Various embodiments are described, further including: receiving, by the GUI, a second suggested care plan elements; providing, by the GUI, a second predicted value of the KPI and a second care plan cost associated with the second suggested care plan elements using the KPI model;

presenting, by the GUI, the second predicted KPI value and second care plan cost on the GUI; and adding, by the GUI, the second predicted KPI value and the second cost to the plot.

Various embodiments are described, further including: receiving, by the GUI, an input to optimize the care plan; receiving, by the GUI, care plan constraints; producing, by the processor, an optimized care plan based upon the plan constraints, the KPI model, and the patient data; and presenting, by the processor, the optimized care plan on the GUI.

Further various embodiments relate to a care plan system, including: a key performance indicator (KPI) model configured to predict the value of a KPI for a specific patient based upon patient data and care plan elements; cost data indicating the cost of the care plan elements; a care plan optimizer configured to determine an optimized care plan and the cost of the optimized care plan based upon care plan constraints, the KPI model, the cost data, and patient data; and a graphical user interface (GUI) configured to receive the care plan constraints and to present an optimized predicted KPI value and optimized care plan cost.

Various embodiments are described, wherein the KPI model is a linear or logistic regression model.

Various embodiments are described, wherein the GUI presents a plot of cost versus the predicted KPI value for the optimized KPI value and optimized cost.

Various embodiments are described, wherein the GUI is configured to receive suggested care plan elements, to provide a predicted value of the KPI and a care plan cost associated with the suggested care plan elements using the KPI model, to present the predicted KPI value and care plan cost, and to add the predicted KPI value and the cost to the plot.

Further various embodiments relate to a method for generating care plan, including: receiving first care plan constraints; producing, by a processor, a first optimized care plan based upon the first plan constraints, the KPI model, and patient data; producing, by the processor, a first optimized KPI value and first optimized cost indicating the cost of the optimized care plan; presenting, by the processor, the first optimized care plan, first optimized KPI value, and first optimized cost on the GUI.

Various embodiments are described, wherein the KPI model is a linear regression model.

Various embodiments are described, further including presenting by the GUI a plot of cost versus the predicted KPI value for the first optimized KPI and first optimized cost.

Various embodiments are described, further including: receiving, by the GUI, second care plan constraints; producing, by a processor, a second optimized care plan based upon the second plan constraints, the KPI model, and patient data; producing, by the processor, a second optimized KPI value and second optimized cost indicating the cost of the second optimized care plan; and

presenting, by the processor, the second optimized care plan, second optimized KPI value, and second optimized cost on the GUI; and adding the second optimized KPI value and the second optimized cost to the plot.

Various embodiments are described, further including: receiving, by the GUI, an input to manually enter a care plan; receiving via a graphical user interface (GUI) care plan elements; predicting a second value of a key performance indicator (KPI) using a KPI model for a specific patient based upon patient data and the first care plan elements; producing second cost indicating the cost of the care plan elements; and presenting the second predicted KPI value and second cost the on the GUI.

### BRIEF DESCRIPTION OF THE DRAWINGS

In order to better understand various exemplary embodiments, reference is made to the accompanying drawings, wherein:

FIG. 1 illustrates a first view in the graphical user interface (GUI) where a care manager inputs care plan data to determine a predicted KPIs;

FIG. 2 illustrates a first view in the GUI where a care manager inputs care plan constraints to determine an optimized care plan meeting the constraints;

FIG. 3 illustrates a method for a care manager using the treatment plan system to determine an optimized care plan within certain plan constraints; and

FIG. 4 illustrates a method for a care manager using the treatment plan system to determine a care plan based upon a manual input of care plan elements by the care manager.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Currently, there is no evidence-based protocols for care planning. Different patients may get the same care plan although they have different needs, while patients with same needs may get different plans. In the embodiments described herein, a tool is described that assists care managers to develop individual care plans based on historic data.

The treatment plan system may include a database module that contains historic data of patients. The database may include the following data for each patient: data on the patient's medical condition; key performance indicators (KPIs) data, for example, re-admission; annual cost; lab results, and so on; data on care plans, for example, home visits, phone calls, assistance with transportation, and so on; and a table or other data structure with cost of each element of the care plan.

The treatment plan system also includes a machine learning module that creates a KPI prediction model for each KPI based on the provided patient data. For example, if the KPI is probability to be admitted in the next year, then the prediction model may be

Prob(admission)=0.2*age+0.1*if_not_married+0.1*lab_result+0.3*John hopking index+0.1*# of home visits+0.05*# of phone calls+0.1*# of follow up appointment

where age, if_not_married, lab_result and John hopking index are the patient characteristics while # of home visits, and # of phone calls are the care plan elements, which are controlled by the care manager. The prediction module may predict a value of the selected KPI based on data of a new patient.

The treatment plan system also includes a graphical user interface (GUI) that allows care managers to explore different plans. The GUI will allow the care manager to explore different care plan options and will present KPI prediction(s) and the cost for each care plan option. The GUI will also allow care managers to compare different options, so the care manager may choose the plan that best fits the patient based on cost.

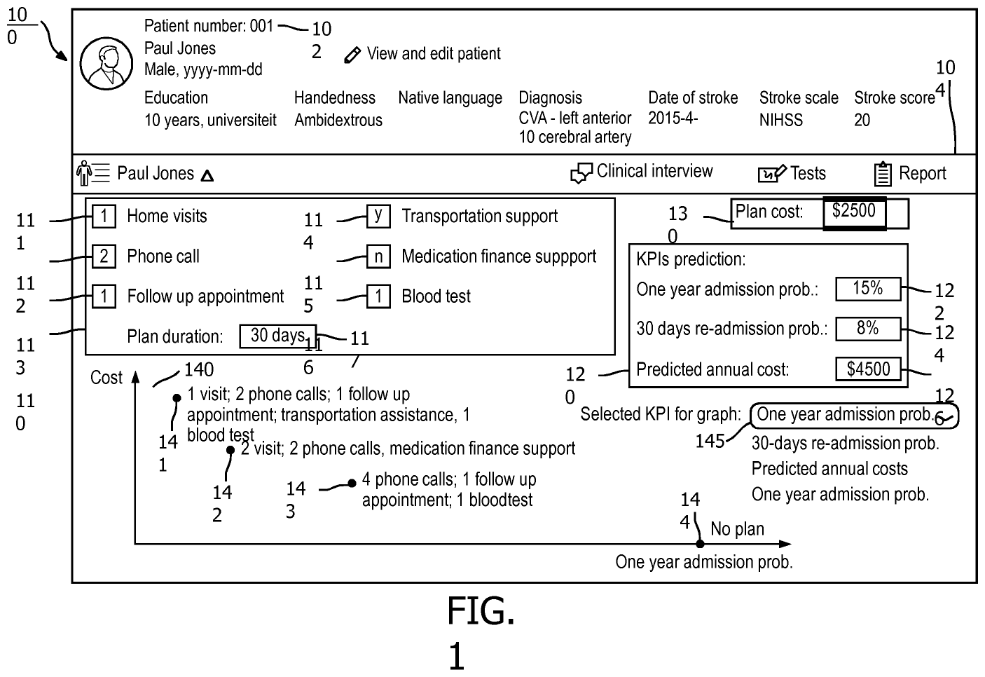

FIG. 1 illustrates a first view in the GUI where a care manager inputs care plan data to determine a predicted KPIs. The GUI 100 may include a patient information panel 102, a care plan panel 110, a predicted KPI panel 120, a plan cost panel 130, and plan graph panel 140.

The patient information panel 102 may include various patient information such as name, patient number, gender, birth date, education, handedness, native language, diagnosis, and in this example of a stroke, date of stroke, stoke scale, and stroke score. Various other information may be presented as well and may vary based upon the patient's particular diagnosis or condition. The patient panel may also include a tool bar 104 where the patient being viewed may be selected or changed, or more specific additional information for the patient may be obtained by clicking on icons to retrieve, for example, a clinical interview, tests, and reports.

The care plan panel 110 is where a care plan manager enters suggested care plan details (e.g., one home visit, two phone calls and so on). The various available care plan details may include the number of home visits 111, the number of phone calls 112, the number of follow up appointments 113, transportation support 114, medication finance support 115, number of blood tests 116, and the care plan duration 117. Other plan details may be included as well based upon what aspects of a care plan are important and may be adjusted by the care manager. Also, the specific care plan details may be tailored to each patient, their preferences, insurance coverage, diagnosis, etc.

The predicted KPI panel 120 may include various KPIs of interest, for example, a one-year admission probability 122, a 30-days re-admission probability 124, and a predicted annual cost of healthcare services provided to the patient 126. Such KPIs may be specific to the patient, to the patient's diagnosis or condition, a recent medical event, or may be selected by the care plan manager. As will be described below, a KPI may be developed that is a combination of other KPIs, for example a weighted combination of a number of KPIs.

The plan cost panel 130 presents the cost of the chosen care plan.

The plan graph panel 140 may present a plot of a specific KPI versus cost (or another KPI if desired). As the care plan manager tries different care plans, the resulting cost and KPI value may be plotted. This allows a care plan manger to see how the various care plans affect the KPI values. Four different care plans are plotted. Care plan 141 includes one visit, two phone calls, one follow up appointment, transportation assistance, and one blood test. Care plan 142 includes two visits, two phone calls, and medication finance. Care plan 143 includes four phone calls, one follow up appointment, and one blood test. Care plan 144 is no care plan at all. Further, a drop-down menu 145 may be used by the care plan manager to select different KPIs for the graph. It is noted that may other sorts of graphs and plots may displayed showing the variation in various KPIs based upon different care plans. Also, a combined KPI may be displayed that combines a set of KPIs. Also, a second vertical axis may be used to plot another KPI versus the KPI along the horizontal axis.

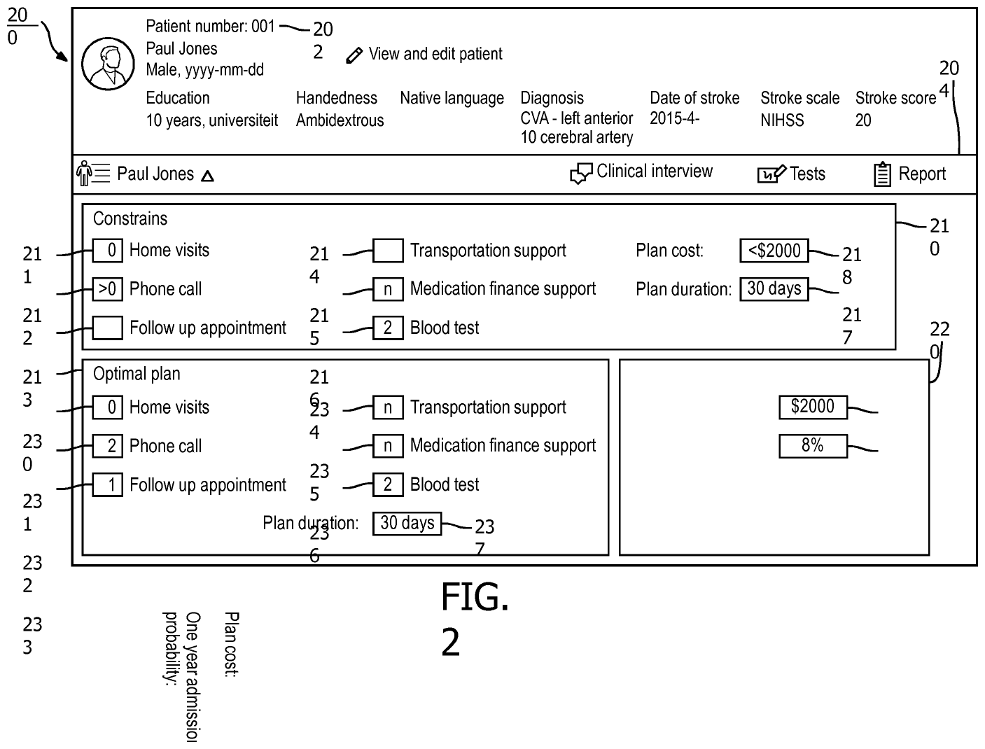

FIG. 2 illustrates a first view in the GUI where a care manager inputs care plan constraints to determine an optimized care plan meeting the constraints. The GUI 200 may include a patient information panel 202, a care plan constraints panel 210, a predicted optimized KPI panel 220, and an optimized care plan cost panel 230.

The patient information panel 202 may include various patient information such as name, patient number, gender, birth date, education, handedness, native language, diagnosis, and in this example of a stroke, date of stroke, stoke scale, and stroke score. Various other information may be presented as well and may vary based upon the patient's particular diagnosis or condition. The patient panel may also include a tool bar 204 where the patient being viewed may be selected or changed, or more specific additional information for the patient may be obtained by clicking in icons to retrieve for example, a clinical interview, tests, and reports.

The care plan constraints panel 210 is where a care plan manager enters care plan constraints (e.g., zero home visits, at least one phone call, medication finance support, two blood tests, a cost limitation of $2,000, and a plan duration of 30 days). The various available care plan constraints may include the number of home visits 211, the number of phone calls 212, the number of follow up appointments 213, transportation support 214, medication finance support 215, number of blood tests 216, care plan cost 218, and the care plan duration 217. Other plan details may be included as well based upon what aspects of a care plan are important and may be constrained by the care manager. Also, the specific care plan constraints may be tailored to each patient, their preferences, insurance coverage, diagnosis, etc. If such patient preferred or other patient related constraints are known, they may be populated in the associated fields.

The optimized care plan panel 230 is where an optimized care plan based upon care plan constraints is shown (e.g., zero home visits 231, two phone calls 232, one follow up visit 233, no transportation support 234, no medication finance support 235, two blood tests 236, and a plan duration of 30 days 237).

The predicted optimized KPI panel 220 may include various KPIs of interest, for example, a plan cost 224 and a one-year admission probability 222. Such KPIs may be specific to the patient, to the patient's diagnosis or condition, a recent medical event, or may be selected by the care plan manager. A KPI may be developed that is a combination of other KPIs, for example a weighted combination of a number of KPIs. Although, not shown, a plot similar to the plot of FIG. 1 may also be shown, especially if there are other care plans previously explored to which the optimized care plan may be compared.

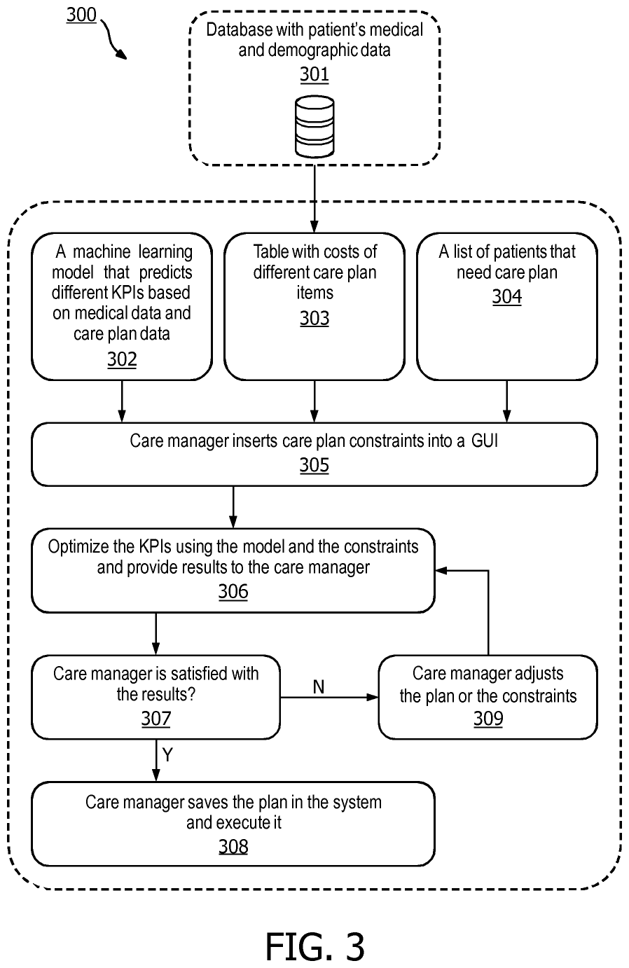

FIG. 3 illustrates a method for a care manager using the treatment plan system to determine an optimized care plan within certain plan constraints. The treatment plan system includes a database 301 with a patient's medical and demographic data and cost data that is used to define models for predicting KPIs 302 for patients. The machine learning models for the KPIs 302 may be determined using patient training data and linear regression to produce a model for the KPI of interest. An example of such a model was presented above. A table of cost items 303 for different care plan elements is also extracted from the data base 301. Also, a list of patients needing a care plan may be developed as well 304. With the KPI models 302, cost table 303, and list of patients 304 in place, the care manager may use the GUI to input care plan constraints 305.

Next, the care plan system may optimize the KPIs 306 using the KPI models 302 and constraints input by the care manager. A care plan optimizer may use logistic regression to optimize a KPI based upon the KPI model and constraints. The logistic regression formula is:

$$Prob ( admission ) = \frac{1}{1 + {e}^{( z )}} where z = 0.2 * age + 0.1 * if\_not \_married + 0.1 * lab\_result + 0.3 * John hopking index + 0.1 * \# of home visits + 0.05 * \# of phone calls + 0.1 * \# of follow up appointment$$

The probability of admission may be optimized as follows. The number of times that each of the three care items is used may be denoted by x₁, x₂ and x₃. Let Cᵢ be the cost of intervention i and C be the maximum cost. The optimization problem is

$$Min \frac{1}{1 + {e}^{( z )}}$$

### Subject to

$${x}_{1} = 0 , 1 , 2 , 3 , … for i = 1 , 2 , 3 \\ {\sum }_{i = 1}^{3} {C}_{i} * {x}_{i} < C$$

Once the optimized KPI is determined it is presented to the care manager 306. The care manager then determines if they are satisfied with the results of the optimization. If not, the care plan manager may manually adjust the care plan elements or enter new care plan constraints 309 and determine a new value for the KPIs 306. This process may be repeated until the care manager has settled on a care plan for the patient. Then the care manager may save the care plan and then execute the care plan 308. The care plan system may use the first and second GUIs of FIGS. 2 and 3 to receive data from the care manager and present the results to the care manager.

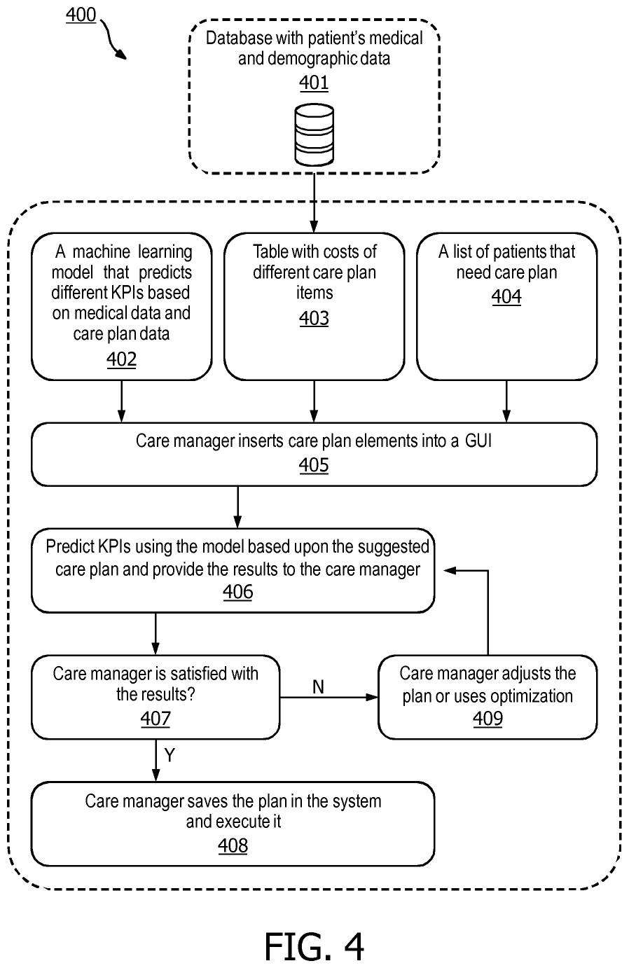

FIG. 4 illustrates a method for a care manager using the treatment plan system to determine a care plan based upon a manual input of care plan elements by the care manager. The treatment plan system includes a database 401 (which is the same database 301 of FIG. 3) with a patient's medical and demographic data and cost data that is used to define models for predicting KPIs 402 for patients (which is the same as model 302 in FIG. 3). The machine learning models for the KPIs 402 may be determined using patient training data and linear regression to produce a model for the KPI of interest. An example of such a model was presented above. A table of cost items 403 for different care plan elements (which is the same as the table 303 in FIG. 3) is also extracted from the data base 401. Also, a list of patients needing a care plan may be developed as well 404 (which is the same as the list of patients 304 in FIG. 3). With the KPI models 402, cost table 403, and list of patients 404 in place, the care manager may use the GUI to input suggested care plan elements 405.

Next, the care plan system may predict the KPIs 406 using the KPI models 402 and suggested care plan elements input by the care manager. Once the predicted KPI is determined it is presented to the care manager 406. The care manager then determines if they are satisfied with the results of the suggested care plan. If not, the care plan manager may manually adjust the care plan elements and determine a new value for the KPIs or the care manager may enter care plan constraints 409 to produce optimized KPIs. This process may be repeated until the care manager has settled on a care plan for the patient. Then the care manager may save the care plan and then execute the care plan 408. The care plan system may use the first and second GUIs of FIGS. 2 and 3 to receive data from the care manager and present the results to the care manager.

The care plan system described herein provides a technological solution to providing patients with individualized care plans. The use of patient data to develop and train models of KPIs provides the tools for a care giver to develop individualized care plans for each patient based upon specific needs of the patient. Because the KPI models are trained using patient data, they may accurately predict the KPIs of interest to the care manager. Such a care plan system leads to improved evidence-based care plans for a wide variety of patients as opposed to existing more rigid care plans being applied to each patient, or each care manager providing different care plans from one another.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, computer servers, desktop or laptop computers, tablets, mobile phones, or other similar devices.

The memory may include various memories such as, for example L1, L2, or L3 cache or system memory. As such, the memory may include static random access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems.

For example, the care plan system may be implemented as software on a server, a specific computer, on a cloud computing, or other computing platform. This software then provides the care manager the GUI, for example, a web-based interface, stand-alone software, other methods on the devices used by the care manager. The devices used by the care manager may be any type of computing device, for example, desktop computers, laptop computers, tablets, smart phones, smart watches and wearable devices, etc., capable of interacting with or hosting the software implementing the care plan system. Also, the care plan system may be implemented on any of types of devices used by the care manager.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A care plan system, comprising:
a key performance indicator (KPI) model configured to predict the value of a KPI for a specific patient based upon patient data and care plan elements;
cost data indicating the cost of the care plan elements; and
a graphical user interface (GUI) configured to receive first suggested care plan elements, to provide a first predicted value of the KPI and a first care plan cost associated with the first suggested care plan elements using the KPI model, and to present the first predicted KPI value and first care plan cost.

2. The care plan system of claim 1, wherein the KPI model is a linear or logistic regression model.

3. The care plan system of claim 1, wherein the GUI presents a plot of cost versus the predicted KPI value for the first predicted KPI and first cost.

4. The care plan system of claim 3, wherein the GUI is configured to receive second suggested care plan elements, to provide a second predicted value of the KPI and a second care plan cost associated with the second suggested care plan elements using the KPI model, to present the second predicted KPI value and second care plan cost, and to add the second predicted KPI value and the second cost to the plot.

5. A method for generating care plan, comprising:
receiving, via a graphical user interface (GUI), first care plan elements;
predicting, by a processor, a first value of a key performance indicator (KPI) using a KPI model for a specific patient based upon patient data and the first care plan elements;
producing first cost data indicating the cost of the care plan elements; and
presenting the first predicted KPI value and first care plan cost the on the GUI.

6. The method of claim 5, wherein the KPI model is a linear or logistic regression model.

7. The method of claim 5, further comprising presenting by the GUI a plot of cost versus the predicted KPI value for the first predicted KPI and first cost.

8. The method of claim 7, further comprising:
receiving, by the GUI, a second suggested care plan elements;
providing, by the GUI, a second predicted value of the KPI and a second care plan cost associated with the second suggested care plan elements using the KPI model;
presenting, by the GUI, the second predicted KPI value and second care plan cost on the GUI; and
adding, by the GUI, the second predicted KPI value and the second cost to the plot.

9. The method of claim 5, further comprising:
receiving, by the GUI, an input to optimize the care plan;
receiving, by the GUI, care plan constraints;
producing, by the processor, an optimized care plan based upon the plan constraints, the KPI model, and the patient data; and
presenting, by the processor, the optimized care plan on the GUI.

10. A care plan system, comprising:
a key performance indicator (KPI) model configured to predict the value of a KPI for a specific patient based upon patient data and care plan elements;
cost data indicating the cost of the care plan elements;
a care plan optimizer configured to determine an optimized care plan and the cost of the optimized care plan based upon care plan constraints, the KPI model, the cost data, and patient data; and
a graphical user interface (GUI) configured to receive the care plan constraints and to present an optimized predicted KPI value and optimized care plan cost.

11. The care plan system of claim 10, wherein the KPI model is a linear or logistic regression model.

12. The care plan system of claim 10, wherein the GUI presents a plot of cost versus the predicted KPI value for the optimized KPI value and optimized cost.

13. The care plan system of claim 12, wherein the GUI is configured to receive suggested care plan elements, to provide a predicted value of the KPI and a care plan cost associated with the suggested care plan elements using the KPI model, to present the predicted KPI value and care plan cost, and to add the predicted KPI value and the cost to the plot.

14. A method for generating care plan, comprising:
receiving first care plan constraints;
producing, by a processor, a first optimized care plan based upon the first plan constraints, the KPI model, and patient data;
producing, by the processor, a first optimized KPI value and first optimized cost indicating the cost of the optimized care plan;
presenting, by the processor, the first optimized care plan, first optimized KPI value, and first optimized cost on the GUI.

15. The method of claim 14, wherein the KPI model is a linear regression model.

16. The method of claim 14, further comprising presenting by the GUI a plot of cost versus the predicted KPI value for the first optimized KPI and first optimized cost.

17. The method of claim 16, further comprising:
receiving, by the GUI, second care plan constraints;
producing, by a processor, a second optimized care plan based upon the second plan constraints, the KPI model, and patient data;
producing, by the processor, a second optimized KPI value and second optimized cost indicating the cost of the second optimized care plan; and
presenting, by the processor, the second optimized care plan, second optimized KPI value, and second optimized cost on the GUI; and
adding the second optimized KPI value and the second optimized cost to the plot.

18. The method of claim 14, further comprising:
receiving, by the GUI, an input to manually enter a care plan;
receiving via a graphical user interface (GUI) care plan elements;
predicting a second value of a key performance indicator (KPI) using a KPI model for a specific patient based upon patient data and the first care plan elements;
producing second cost indicating the cost of the care plan elements; and
presenting the second predicted KPI value and second cost the on the GUI.
